(19) United States
(12) Patent Application Publication
(10) Pub. No.: US 2018/0372734 A1
(43) Pub. Date: Dec. 27, 2018

(54) LATERAL FLOW IMMUNOASSAY DEVICE

(71) Applicant: AESD RESEARCH & DEVELOPMENT GMBH, Berlin (DE)

(72) Inventors: Stefan PFENNINGER, Berlin (DE); Allan GAERTKE, Dubai (AE)

(21) Appl. No.: 15/771,132

(22) PCT Filed: Oct. 26, 2015
(86) PCT No.: PCT/EP2015/074729

§ 371 Date: Apr. 26, 2018

(51) Int. Cl. G01N 33/543; G01N 33/52; B01L 3/00

(52) U.S. Cl. CPC G01N 33/54366; G01N 33/521; B01L 2200/10; B01L 2300/0861; B01L 2200/028; B01L 3/502715

(57) ABSTRACT

The present invention refers to a Lateral Flow Immunoassay (LFIA) test consumable and a corresponding reader device. The LFIA test consumable (10) comprises a casing (12) and two or more LFIA test strips (14), which are assembled on different side surfaces (A . . . D) of the casing (12) is provided. The corresponding reader device (20) for the LFIA test consumable (10) comprises a casing (22) and a measurement module (30) being assembled within the casing (22) and including (i) a measurement channel (32) adapted for accepting the LFIA test consumable (10); (ii) two or more optical units (34a . . . 34d) assembled on different side surfaces (A′ . . . B) of the measurement channel (32), each optical unit (34a . . . 34d) comprising at least one light source (36a . . . 36d) and at least one light sensor (38a . . . 38b) adapted for providing an optical signal at a signal output; and (iii) an evaluation module (40) being assembled within the casing (22), connected to the signal output of each optical unit (34a . . . 34d) and adapted for evaluating the test results of an LFIA test consumable (10). Further, a process for LFIA testing is provided.

The present invention refers to a Lateral Flow Immunoassay (LFIA) test consumable and a corresponding reader device. Further, a process for LFIA testing is provided.

### TECHNOLOGICAL BACKGROUND

Lateral Flow Immunoassay (LFIA) tests represent a proven technology applied to a wide variety of Point-of-Care (POC) testing technologies and other field use applications. LFIA is a mature technology that is easy to manufacture, requires only minimal operator-depended steps and interpretation, enjoys high market acceptance, has a long shelf-life (usually around 24 months), operates with only small volumes of sample fluid and yet provides high sensitivity, specificity, and stability making it ideal to integrate with onboard electronics, reader systems, and information systems.

Typical LFIA tests yields a yes/no answer, although with the help of multiplexing and multiple capture lines engineered with different cut-offs, semi-quantitative results become possible without comprising on result reliability or over-complicating the user-experience.

Multiplexing LFIA test strips is however complicated and so far, more than four lines per LFIA test strip compromises the quality of the performance. Other systems like those using microarray multiplex lab-on-a-chip (LOC) technology that assays large amounts of biological material using high-throughput screening miniaturized, multiplexed and parallel processing and detection methods are far from being available for the general public. The current lack of a concept for multiplexed LFIA test consumable not only makes the usability of the above-mentioned semi-quantitative method complicated, but also renders the simultaneous analysis of multiple markers impossible.

For a wide variety of medical and environmental conditions, different systems for LFIA test exist, most of which make use of an optoelectronic reading device to analyze the test's analyte regions and present results using a display. To be cost effective, a large group of these devices make use of inexpensive detection systems based on the interplay of photo diodes and light emitting diodes, which produces highly accurate results and at the same time drastically reduces costs compared to optical detectors and supporting electronics required in typical image analysis. The design of such detector systems, based on photodetectors and light emitting diodes, are either static, where each captures line in the analyte region of an LFIA test consumable is being evaluated separately by immobile detectors, or dynamic, where a one-dimensional profile of the optical signals of said analyte region is generated.

Both the static and the dynamic systems suffer from severe drawbacks: static systems, measuring only absolute color values, not only lack the relative measuring reference but are in particular lacking scalability, meaning once such a device is manufactured, only a test with one particular geometry of analyte regions can be analyzed. Dynamic systems on the other hand include either electro-mechanical parts, which render the device fragile and expensive, or perform the measuring during insertion/ejection of LFIA test consumables into the device.

Systems executing the latter type of reading lack location information and can therefore only mutually relate and compare the detected maxima, which by itself render a reliable analysis of multiplexed LFIA test consumables impossible and makes an improved technology urgently needed.

EP 2453242 A1 discloses a prior art method and system utilizing a multiplexed multi-line LFIA test device with integrated quality assurance label. A typical prior art optoelectronic reader device for LFIA testing is disclosed by U.S. 2005/0208593 A1.

The objective problem of the invention is therefore related to the problem of providing novel miniaturized multiplexed LFIA test consumables, a corresponding reader device, and unified process for LFIA testing, enabling the simultaneous performing/reading of a plurality of markers/test lines at a plurality of analyte regions, as well as allowing semi-quantitative analysis while maintaining the high reliability of the established LFIA technology and its simple handling.

### SUMMARY OF INVENTION

The invention solves the objective problem by at least providing a LFIA test consumable as defined in claim 1 as well as a corresponding reader device as defined in claim 9.

The inventive LFIA test consumable comprises a casing and two or more LFIA test strips, which are assembled on different side surfaces of the casing.

The corresponding reader device for the LFIA test consumable comprises a casing a measurement module being assembled within the casing and including
(i) a measurement channel adapted for accepting the LFIA test consumable;
(ii) two or more optical units assembled on different side surfaces of the measurement channel, each optical unit comprising at least one light source and at least one light sensor adapted for providing an optical signal at a signal output; and
(iii) an evaluation module being assembled within the casing, connected to the signal output of each optical unit, and adapted for evaluating the test results of an LFIA test consumable.

The invention is based on the finding that an highly effective miniaturization can be achieved by utilizing the full available surface area of a LFIA test consumable not only within a single plane but onto a few up to all side surfaces of a corresponding carrier for a set of individual

LFIA test strips. This means, for an effective two-dimensional carrier system (card- or strip-type) an upper and lower side of the carrier can be equipped with individual LFIA test strips. In a triangular carrier system with rod-like structure three side surfaces are available for assembling the LFIA test strips. Therefore, in a square shaped carrier system four side surfaces can be used for the assembling, and so on. It is also part of the present invention that the casing allows an easy manufacture and distribution for the producers of the LFIA test consumables as well as to provide an easy handling, storing and processing scheme for the costumers of those consumables by providing a rigid and stable platform for performing LFIA testing. The corresponding reader device allows simultaneous reading of all side surfaces of the LFIA consumable.

The casing of the LFIA test consumable may comprise a protective cap system covering viewing portions of said casing during liquid sampling. The cap system may further protect said viewing portions during handling and storage. A viewing portion may be a region of the casing where the LFIA test lines or the analyte regions are observable on the various sides of the casing. The casing may also comprise transparent viewing windows over the different viewing portions alone or in combination with said protective cap system.

The assembled LFIA test strips may comprise, apart from one indispensable control line per LFIA test strip, multiple capture lines engineered with different cut-offs for semi-quantitative results or multiple capture lines for an inner-strip multiplexing of the detection. By combining widely approved and accepted pre-existing technologies within an LFIA test consumable of the present invention, a miniaturized, yet reliable, user-friendly and secure product is guaranteed.

In a preferred embodiment of the LFIA test consumable, the LFIA test consumable includes a common sample pad for the LFIA test strips while in another embodiment each or some of the LFIA test strips include their own sample pads. An LFIA test consumable corresponding to the present invention thus may comprise one or more sample pads. A sample pad is required to collect test fluid by pipette drops and/or by urine midstream and supply the individual LFIA test strips with sample fluid. By providing a common sample pad for the multiplexed LFIA test consumable single application of test fluid is necessary thus reducing the required amount of test fluid and enhancing ease of application. The common sample pad can be on top of one or all of the side surfaces of the LFIA test consumable. It may be combined in the form of a common wetting pad. The sample pad can further be on the top of an LFIA test consumable in the form of a plunge pad. For a card-type LFIA test consumable one of the card-edges may be immersible into a test liquid to absorb a sufficient amount of test liquid for allowing LFIA testing.

The common sample pad may be protected from the environment by a suitable shielding or protective cap system to avoid contaminations of said sample pad or any potentially dangerous exposure of medical personal or other individuals to residual test liquid.

In a particularly preferred embodiment, the LFIA test consumable includes 2 through 6 individual LFIA test strips. These are either each assembled on different side surfaces of a corresponding casing with up to hexagonal shape or several of these individual LFIA test strips are assembled on a single surface thus reducing the total number of required surfaces. For example, 6 individual LFIA tests could be assembled on a strip-type casing as 3 individual test strips per side surface or on a triangular casing as 2 individual test strips per side surface.

In another preferred embodiment the LFIA test consumable further includes one or more identification units adapted for at least providing an individual reference for the type of LFIA test strips provided on the LFIA test consumable. For example, these identification units may without any limitation comprise specific color coding schemes, simple identification marks for visual inspection, radio-frequency identification (RFID), or a means for electronic chipping.

In another preferred embodiment of the LFIA test consumable, the identification unit is further adapted for providing at least one of producer, supplier, medical instance, date of production, date of storage life, production batch number, information of the orientation of the LFIA test consumable, alignment information about the relative position of LFIA tests and interpretation range. The information encoded to the identification unit may be used to provide identification about the supplier and the logistical and pharmaceutical data of the LFIA test consumable. It may also be used to provide an individualization of single LFIA test consumables for allowing a distinction between different identical LFIA tests which are performed within one test series. Furthermore, the identification unit may provide information on the alignment of the individual LFIA test strips on the LFIA test consumable, the exact number and the spatial positions of the various LFIA test lines, the specific type or subclass of test and the corresponding interpretation ranges, for example, in interpreting the brightness, hue, and/or color of the LFIA test lines.

In another preferred embodiment of the LFIA test consumable, each of the LFIA tests is dedicated to a corresponding identification unit being assembled on the same side surface of the LFIA test consumable. In yet another preferred embodiment of the LFIA test consumable, only the LFIA tests assembled on the same side surface are dedicated to a corresponding identification unit which is also assembled on the same side surface of the LFIA test consumable. The identification unit may comprise additional reference lines for calibration.

In a particularly preferred embodiment of the LFIA test consumable, the provided identification unit comprises a barcode which can be electronically analyzed by visual machine inspection. Preferably the bar code is directly related to an individual LFIA test strip. More preferably it is located with the related LFIA test strip on the same side surface of the casing of the LFIA test consumable. For enhancing the amount of information and increase density, the barcode of the present invention can be replaced by a two-dimensional Quick Response Code (QRC), Micro-QR-Code (MQRC), Secure-QR-Code (SQRC), iQR-Code or Frame QR Code which can also be analyzed by machine vision.

In another preferred embodiment of the LFIA test consumable, at least one of the side surfaces and/or at least one of the edges between these side surfaces comprises a means for alignment. This means for alignment can be, without limitation, a specific pin or pin arrangement, a nose, notch, slide or splint which is arranged on the casing to mark a specific spatial position or direction for handling the LFIA test consumable. In other words, the means for alignment can provide information about a specific alignment of the LFIA test consumable in respect to other equipment or device.

Especially in field use applications, the successful utilization of the various available prior art LFIA test consumables is sometimes difficult or cumbersome due to the differently wide interpretation ranges, various indication methods, erroneous result evaluation, complex and complicated LFIA testing procedures, insufficient documentation abilities and the extremely short test result lifetime. Therefore, a user-friendly electronic reader device must be able to automatically measure, interpret and document all processed LFIA tests without the requirement of time-consuming and error-prone calibration and setting procedures. In combination with a corresponding set of reliable and self-setting LFIA test consumables, highly accurate automatized LFIA test readings with detailed analysis of the detected line maxima, their location, their test-type, and their batch number can be provided while at the same time allowing for maximal flexibility independent of the number or location of the different analyte regions that are to be analyzed. By eliminating electro-mechanical scanning mechanisms, spring mechanisms and buttons for the user to press, a simple, cost-effective and user-friendly reader device can be provided.

The reader device is designed to provide an intuitive, user-friendly, cost-effective, low power consuming, miniaturized, high accuracy, portable solution to scan multiplexed LFIA test consumables using photo-metric analysis for all analyte regions and identification units and to analyze and store the analysis thereof. The casing may comprise a means to latch a LFIA test consumable to avoid potential high accelerations when the consumable is pulled out.

The reader device can be based on an improved dynamic scanner principle with high mechanical robustness from a rigid and compact design and by mechanically clamping the LFIA test consumables inside the measurement module. Furthermore, the reader device can be based on a flexible aperture system minimizing scattering of light during measurements by minimizing gaps between the multiplexed capture lines of a LFIA test consumable and apertures of the optical units focusing on analyte regions of the LFIA test consumable preferably to a range between 0 mm and 0.25 mm, between 0 mm and 1 mm, or between 0 mm and 5 mm. The flexible aperture system can be mass produced via injection molding.

The measurement module and/or the evaluation module can be comprised by a flex printed circuit board, which can embrace the measurement channel and the optical units, allowing for measuring all sites of a LFIA test consumable simultaneously.

In a preferred embodiment of the reader device for an LFIA test consumable, the reader device further comprises an activation button which is designed to be switched when the LFIA test consumable is completely or partly inserted into the measurement channel. The activation button can be comprised by the measurement module and/or the casing. It is particularly preferred that the activation button is located inside the measurement channel and automatically switched by a complete insertion of the LFIA test consumable into the measurement channel. Preferably, the activation button can be implemented as a push-switch or a pressure switch.

In particularly preferred embodiment of the reader device for an LFIA test consumable, the evaluation module comprises a data storage unit including at least data sets for identification of LFIA test consumables. The data storage unit can be fully implemented inside the reader device by itself comprising a means of non-volatile memory, e.g., an EEPROM or flash memory. It is highly preferred that all the information, which can be gathered from any LFIA test consumable, can be permanently or temporarily stored inside the data storage unit. Beyond data sets for identification of LFIA test consumables and the results of corresponding measurements, the stored data sets may further include at least on of producer, supplier, medical instance, date of production, date of storage life, production batch number, information of the orientation of the LFIA test consumable, alignment information about the relative position of LFIA tests and interpretation range.

In a further particularly preferred embodiment of the reader device for an LFIA test consumable, each data set for a distinct LFIA test consumable includes information on the spatial position of test regions at the LFIA tests being provided on a distinct LFIA test consumable. This can include a specific spatial range where the multiplexed capture lines of the distinct LFIA test consumable can be expected.

In another preferred embodiment of the reader device for an LFIA test consumable, each optical unit comprises two light sources and two light sensors adapted for providing two optical signals at a signal output. In another preferred embodiment, at least one optical unit comprises N light sources and N light sensors adapted for providing N optical signals at a signal output, wherein N is greater or equal to 2. The light sources and sensors can be arranged in groups such that for each light source a corresponding light sensor is assigned for detection. One of such groups in an optical unit may be arranged to may be arranged to detect information of the analyte region of a specific LFIA test strip while the other group may be arranged to readout the identification unit of the corresponding LFIA test consumable. An optical separation against direct light exposure or scattered light can be achieved by a vertical offset between two corresponding light sources and light detectors such that one of the elements is elevated against the other. The individual light sources may comprise a build-in lens and an angle of inclination against the corresponding light sensor to minimize scattered light and to improve reading performance while optically measuring the LFIA test consumable. The individual light sensors may further comprise localized cost-effective on-chip amplifiers and data buffer systems to optimize the detected optical signal. The signal output may be directly connected to evaluation module for evaluating the measured optical signals.

In another preferred embodiment of the reader device for an LFIA test consumable, the evaluation module comprises a means for test result and status feedback; and/or means for wireless communication. The means for test result and status feedback may comprise an indicator for at least providing information about one of test result, battery status, device status, measuring process, and evaluation status to a user of the reader device. The indicator can optically or acoustically indicate by comprising a single or multiple light emitting diodes or a speaker device, respectively. The means for test result and status feedback may further comprise a LCD or OLED display for directly providing test result and status feedback information. The means for wireless communication may be based on at least one of Bluetooth, WLAN, GPS, GRPS, UMTS, Edge, 3G, 4G or NFC. It can be designed to directly communicate with ordinary local counterparts (e.g. smartphones, notepads, pads, laptops, computers, or workstations) or via direct connection to the internet or other wide-range communication services with remote clients or cloud-based services.

According to another aspect of the invention, there is provided a process for LFIA testing comprising the steps of: a) providing a LFIA test consumable as defined in one of the claims 1 to 8, and providing a reader device for a LFIA test consumable as defined in one of the claims 9 to 14; b) exposing the sample pad of the LFIA test consumable to a sample fluid; c) pulling the LFIA test consumable into and out of the measurement channel of the reader device, wherein the measurement module is activated and the LFIA test strips of the LFIA test consumable are measured; d) evaluating at least one test result of an LFIA test consumable; and e) presenting a test result and status feedback via the means for test result and status feedback and/or transmitting a test result via the means for wireless communication.

While a user inserts a multiplexed LFIA test consumable in the device, all comprised LFIA test strips can be read at once during insertion and/or ejection of the consumable. The reader device thereby records all maxima in the analyte regions, all reference lines and all information provided by the identification units. The reader device may not comprise any button for the user to press by comprising an automatic LFIA test consumable detection by waking up the reader device with an activation button and subsequently launching the scanning of the LFIA test consumable automatically. The scanned maxima in the analyte regions of individual LFIA test strips can be compared mutually and with respect to the background color of the multiplexed LFIA test consumable. A simultaneous measuring of reference lines may be performed and to provide a detection of local information of maxima in analyte regions, allowing for an unambiguous evaluation of multiplexed LFIA test consumables. The performed device reading and analyzing process guarantees a maximum flexibility regarding the number, absolute and relative position and type of analyte regions.

Further aspects of the invention could be learned from the following description.

### BRIEF DESCRIPTION OF THE DRAWINGS

In the following the invention will be described in further detail. The examples given are adapted to describe the invention.

FIG. 1 shows a perspective view of a LFIA test consumable partly inserted into a corresponding reader device;

FIG. 2 shows a schematic view of a first embodiment of a strip-type LFIA test consumable carrying two LFIA test strips with a first type of sample pad;

FIG. 3 shows a schematic view of a second embodiment of a square shaped LFIA test consumable carrying four LFIA test strips with a first type of sample pad;

FIG. 4 shows a schematic view of a third embodiment of a rod-type LFIA test consumable carrying two LFIA test strips with a second type of sample pad;

FIG. 5 shows a perspective view of the inner parts of a reader device for double-sided LFIA test consumables;

FIG. 6 shows another perspective view of the inner parts of a reader device for double-sided

LFIA test consumables;

FIG. 7 shows a bisected perspective view of a LFIA test consumable partly inserted into a corresponding reader device allowing observing the inner parts of the reader device; and

FIG. 8 shows a process of LFIA testing.

### DETAILED DESCRIPTION OF THE INVENTION

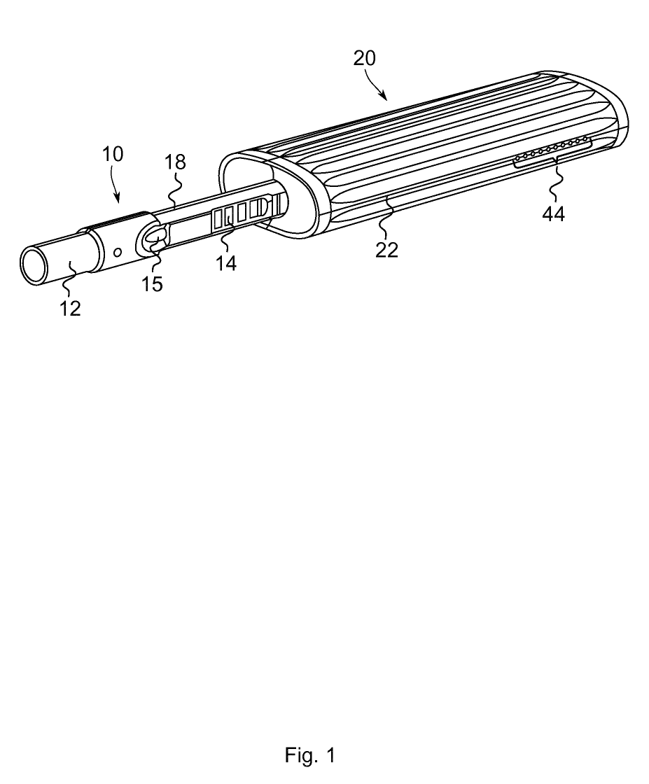

FIG. 1 shows a perspective view of a LFIA test consumable 10 partly inserted into a corresponding reader device 20. The LFIA test consumable 10 comprises a casing 12 for handling. In the given perspective view, the casing 12 shows two openings. These are one opening giving access to a sample pad 15 and one opening to allow an observation of the included LFIA test strip 14. The LFIA test consumable 10 can be fully symmetric in respect to its mid-section or, in case of a single common sample pad 15, the symmetry may be broken by omitting a corresponding opening on the other side of the casing 12. The depicted LFIA test consumable 10 further comprises a means for alignment 18 in the form of a groove here. This alignment groove is supported by a corresponding counterpart, e.g., in the form of a guiding pin, located at the entrance area of the casing 22 of the reader device 20. The casing 22 encloses and protects the inner parts of the reader device 20. It shows no external elements which must be pressed or maintained by a user. There is a line of several LEDs visible on the back section of the reader device 20 which is a form of a direct means for test result and status feedback 44. This means allows giving visually evaluable information in a simple to interpret form to the user. Instead of a LED line, for example, a single low-energy LED or an acoustical feedback could be alternatively applied for user interaction.

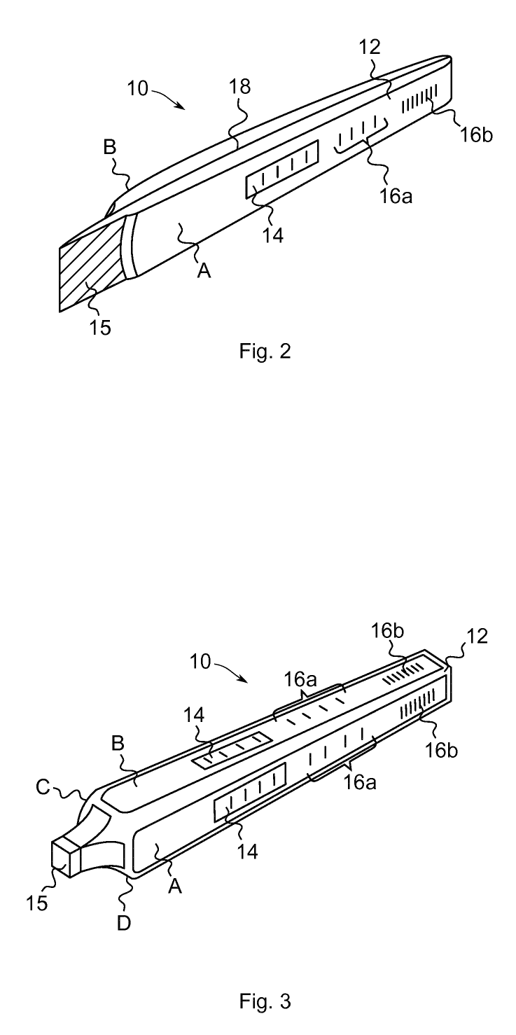

FIG. 2 shows a schematic view of a first embodiment of a strip-type LFIA test consumable 10 carrying two LFIA test strips 14 with a first type of sample pad 15. The sample pad 15 is depicted in the form of a common plunge pad which can be directly inserted with the LFIA test consumable 10 into a test liquid. The plunge pad absorbs the test liquid and moistens the connected LFIA test strips 14 which are located on both side surfaces A, B of the strip-type LFIA test consumable 10. They can be observed by openings in the casing 12 of the LFIA test consumable 10. The depicted LFIA test consumable 10 is fully symmetric to its mid-line. However, there may be differences between the information encoded by the identification units 16 on the two side surfaces A, B. The depicted identification unit 16 on side surface A exemplarily comprises a first section providing reference lines 16a with information about the specific spatial position of the corresponding analyte regions and a barcode 16b which can contain information about LFIA test type, calibration and interpretation ranges, batch number, and so on. The LFIA test consumable 10 further comprises a means for alignment 18 to allow a controlled insertion into a corresponding reader device 20.

FIG. 3 shows a schematic view of a second embodiment of a square shaped LFIA test consumable 10 carrying four LFIA test strips 14 with a first type of sample pad 15. The square shaped carrier system provides four side surfaces A, B, C, D which can be used for the assembling of four individual LFIA test strips 14. However, as the general structure of the LFIA test consumable 10 is comparable to the strip-type design shown in FIG. 2, the corresponding reference signs are transferable. Thereby, the total number of side surfaces is, in principle, merely limited by the actual size of the comprised LFIA test strips 14 and can be increased to a nearly arbitrary number, e.g. six, eight, nine, or ten side surfaces.

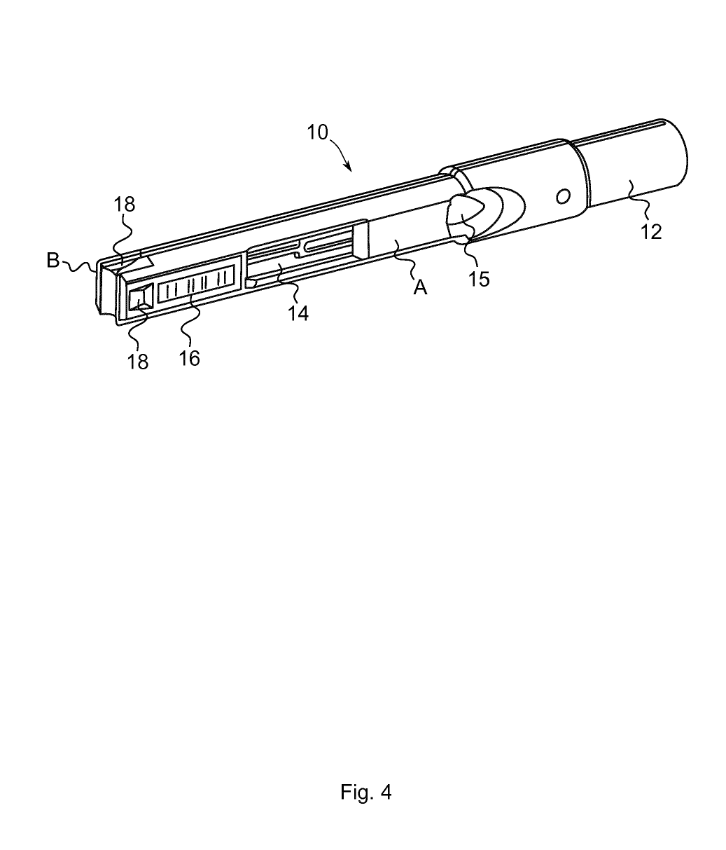

FIG. 4 shows a schematic view of a third embodiment of a rod-type LFIA test consumable 10 carrying two LFIA test strips 14 with a second type of sample pad 15. The reference signs correspond to the assignment in the previous embodiments. The depicted embodiment corresponds to the LFIA test consumable 10 partly shown in FIG. 1. It mainly differs from the embodiment shown in FIG. 2 in that the design of the casing 12 is more circular than strip-type and that the sample pad 15 is of different type having the form of a wetting pad. A wetting pad can be exposed to a test liquid by simply applying single drops from a pipette to its surface. The sample pad 15 can be common and single-sided or for each individual LFIA test strip 14 an individual sample pad 15 is provided on the LFIA test consumable 10. For a simplified and error-prone insertion of the LFIA test consumable 10 in a corresponding reader device 20, the means for alignment 18 is comprised of different alignment aids here. These are, for example and not limiting, multi-lateral guides for merging the LFIA test consumable 10 to a corresponding reader device 20 and guiding notches along the thin upper and lower side surfaces of the LFIA test consumable 10.

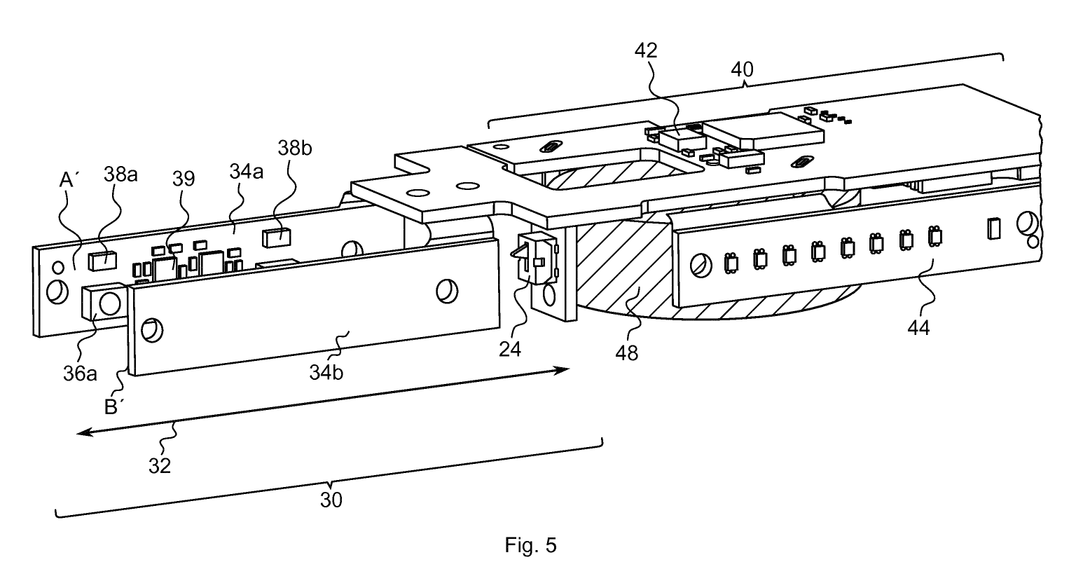

FIG. 5 shows a perspective view of the inner parts of a reader device 20 for double-sided LFIA test consumables 10. This embodiment illustrates a reader device 20 which corresponds to a strip- or rod-type LFIA test consumable 10 carrying two LFIA test strips 14 at maximum. It mainly consists of two different functional sections. These are first a measurement module 30 comprising a measurement channel 32 with two side-surfaces A′, B′ for inserting the LFIA test consumable 10 into it, and second an evaluation module 40 comprising a data storage unit 42 for data storage, a means for test result and status feedback 44 in the form of a LED line, and a lithium battery 48 for energy supply. The measurement module further comprises an activation button 24 at the end of the measurement channel 32, and two optical units 34a, 34b located on each of the side-surfaces A′, B′ of the measurement channel 32 including light sources 36, light sensors 38a, 38b and on-chip amplifiers and data buffer systems 39 for increasing the signal-to-noise ratio of the detection. The depicted measurement channel 32 is provided in total with four light sources 36 and four light sensors 38a, 38b. By that, it is possible to optically readout all provided LFIA test strips 14 of a corresponding LFIA test consumable 10 at once in direct conjunction with optical information which can be provided by two individual identification units 16 located on the two side surfaces of the LFIA test consumable 10. The measured data is then directly processed by the on-chip amplifiers and data buffer systems 39 before being transferred to the evaluation module 40.

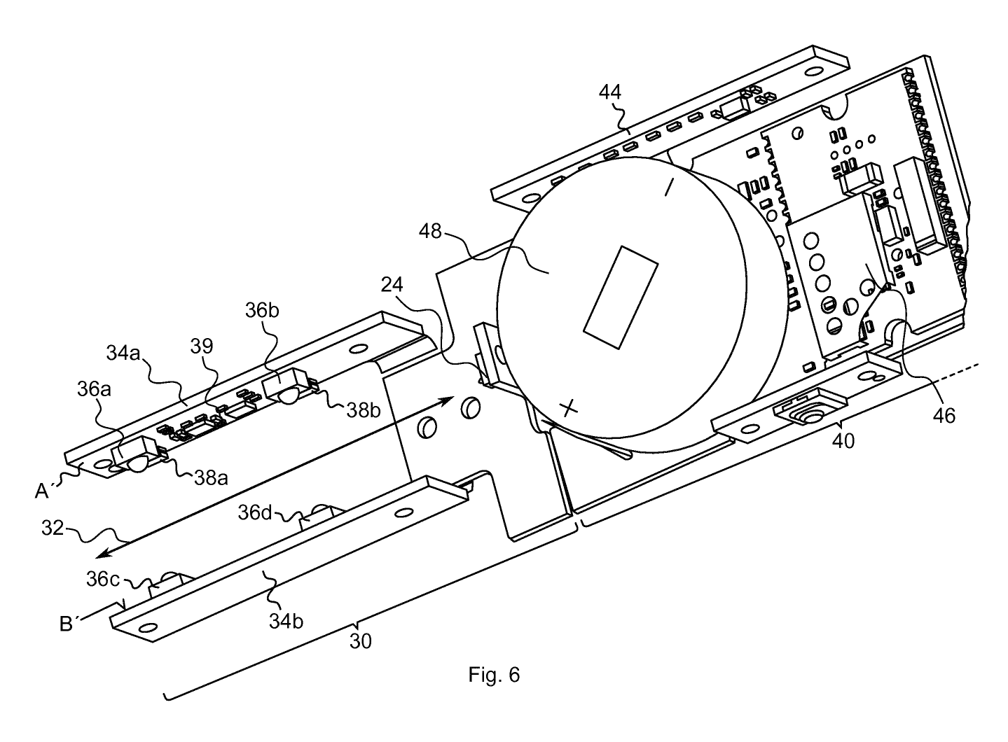

FIG. 6 shows another perspective view of the inner parts of a reader device 20 for double-sided LFIA test consumables 10. The reference signs directly correspond to the reference sign assignment given in FIG. 5. In this view, the identical layout of the two optical units 34a, 34b can be seen. Next to the battery 48 is a means for wireless communication 46 which allows the reader device 20 to interact with an external electronic data reader or device. For example, this reader can be in the form of a mobile device application or an external software program on a notebook or other portable computer.

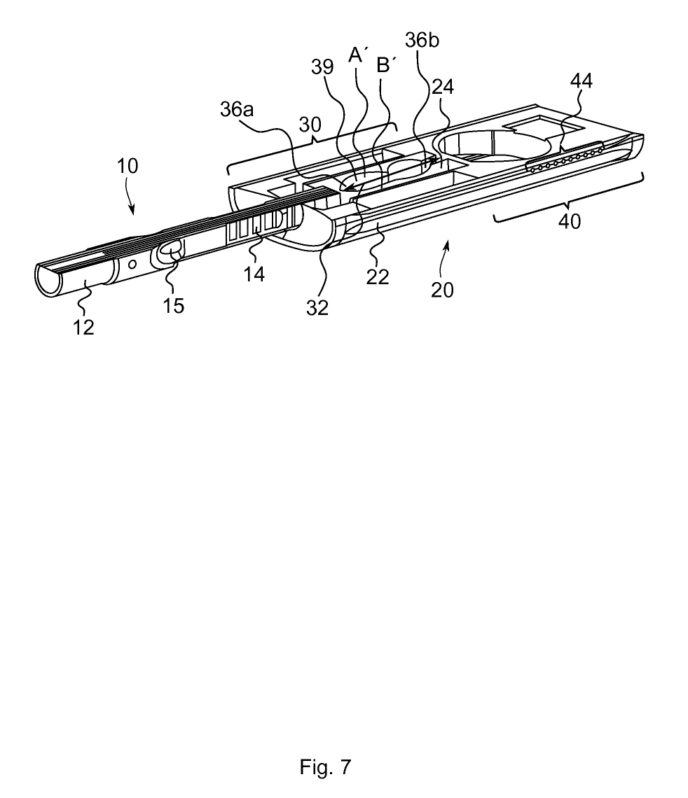

FIG. 7 shows a bisected perspective view of a LFIA test consumable 10 partly inserted into a corresponding reader device 20 allowing observing the inner parts of the reader device. The depicted view corresponds to FIG. 1 and shows elements introduced in FIGS. 4 and 6. The used references sign are identical with the previous assignment made. By pulling the LFIA test consumable 10 into the measurement channel 32 of the reader device 20, the measurement module 30 can be activated by striking the activation button 24 at the end of the measurement channel 32 such that by pulling the LFIA test consumable 10 out of the measurement channel 32 of the reader device 20 the LFIA test strips 14 and the identification units 16 of the LFIA test consumable 10 are optically measured simultaneously. Afterwards, the measurement results are further processed by the on-chip amplifiers and data buffer systems 39 of the optical units 34a, 34b to be further analyzed and evaluated by the evaluation module 40.

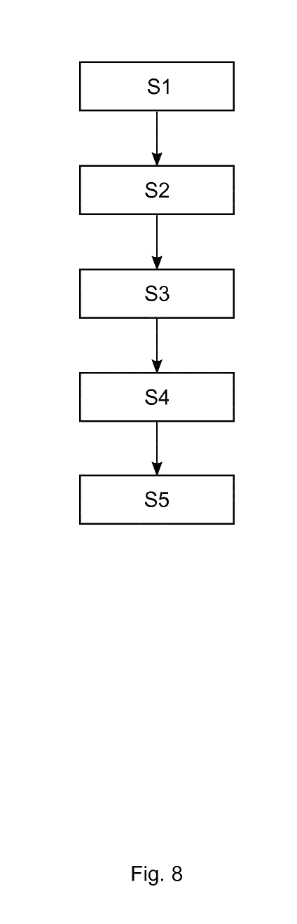

In the following, a process of LFIA testing will be depicted in more detail and with respect to FIG. 8. In S1, a LFIA test consumable 10 and a corresponding reader device 20 are provided. In S2, a sample pad 15 of the LFIA test consumable 10 is exposed to a sample fluid. The form of exposure depends on the specific type of sample pad 15. If it is a common sample pad 15, single sample fluid exposure is sufficient for wetting all LFIA test strips 14 comprised by the LFIA test consumable 10. In S3, the LFIA test consumable 10 is pulled into and out of a measurement channel 32 of the reader device 20. Thereby, an activation button 24 preferably located at the end of the measurement channel 32 may be contacted at the inner pulling turning point such that the reader device 20 is activated and ready for measurement when the pulling out procedure begins. The measurement results are then processed by the on-chip amplifiers and data buffer systems 39 of the optical units 34a, 34b and transferred to the evaluation module 40. In S4, at least one test result of the LFIA test consumable 10 is evaluated by the evaluation module 40. If the LFIA test consumable 10 comprises multiple LFIA test strips 14 for different LFIA tests, other independent test results may be also evaluated. Furthermore, at least semi-quantitative results can be evaluated by providing LFIA test consumables 10 with different sensitivities for a single LFIA test. In S5, a test result and status feedback is presented to a user via a means for test result and status feedback 42 and/or transmitted via a means for wireless communication 44.

### REFERENCE LIST

10 LFIA test consumable
12 casing
14 LFIA test strip
15 sample pad
16 identification unit
16a reference lines
16b barcode
18 means for alignment
20 reader device
22 casing
24 activation button
30 measurement module
32 measurement channel
34a . . . 34b optical units
36a . . . 36d light sources
38a . . . 38b light sensors
39 on-chip amplifiers and data buffer systems
40 evaluation module
42 data storage unit
44 means for test result and status feedback
46 means for wireless communication
48 battery
A . . . D side surfaces (of a LFIA test consumable (10))
A′ . . . B′ side surfaces (of a measurement channel (32))

1. Lateral flow immunoassay (LFIA) test consumable (10) comprising:
a casing (12); and
two or more LFIA test strips (14), which are assembled on different side surfaces (A . . . D) of the casing (12).

2. LFIA test consumable (10) according to claim 1, wherein the LFIA test consumable (10) includes a common sample pad (15) for the LFIA test strips (14), or each or some of the LFIA test strips (14) include their own sample pads.

3. LFIA test consumable (10) according to claim 1, wherein the LFIA test consumable (10) includes 2 through 6 LFIA test strips (14).

4. LFIA test consumable (10) according to claim 1, wherein the LFIA test consumable (10) further includes one or more identification units (16) adapted for at least providing an individual reference for the type of LFIA test strips (14) provided on the LFIA test consumable (10).

5. LFIA test consumable (10) according to claim 4, wherein the identification unit (16) is further adapted for providing at least one of producer, supplier, medical instance, date of production, date of storage life, production batch number, information of the orientation of the LFIA test consumable (10), alignment information about the relative position of LFIA tests (14) and interpretation range.

6. LFIA test consumable (10) according to claim 4, wherein each of the LFIA tests (14) is dedicated to a corresponding identification unit (16) being assembled on the same side surface (A . . . D) of the LFIA test consumable (10).

7. LFIA test consumable (10) according to claim 4, wherein the identification unit (16) compromises a barcode.

8. LFIA test consumable (10) according to claim 1, wherein at least one of the side surfaces (A . . . D) and/or at least one of the edges between these side surfaces (A . . . D) comprises a means for alignment (18).

9. Reader device (20) for an LFIA test consumable (10) comprising:
a casing (22);
a measurement module (30) being assembled within the casing (22) and including
(i) a measurement channel (32) adapted for accepting the LFIA test consumable (10);
(ii) two one or more optical units (34a . . . 34d) assembled on different side surfaces (A′ . . . B′) of the measurement channel (32), each optical unit (34a . . . 34d) comprising at least one light source (36a . . . 36d) and at least one light sensor (38a . . . 38b) adapted for providing an optical signal at a signal output; and
(iii) an evaluation module (40) being assembled within the casing (22), connected to the signal output of each optical unit (34a . . . 34d), and adapted for evaluating the test results of an LFIA test consumable (10).

10. Reader device (20) for an LFIA test consumable (10) according to claim 9, wherein the reader device (20) further comprises an activation button (24) which is designed to be switched when the LFIA test consumable (10) is completely or partly inserted into the measurement channel (32).

11. Reader device (20) for an LFIA test consumable (10) according to claim 9, wherein the evaluation module (40) comprises a data storage unit (42) including at least data sets for identification of LFIA test consumables (10).

12. Reader device (20) for an LFIA test consumable (10) according to claim 11, wherein each data set for a distinct LFIA test consumable (10) includes information on the spatial position of test regions at the LFIA tests being provided on a distinct LFIA test consumable (10).

13. Reader device (20) for an LFIA test consumable (10) according to claim 9, wherein at least one optical unit (34a . . . 34d) comprises N light sources (36a . . . 36d, 36a′ . . . 36d′) and N light sensors (38a . . . 38d, 38a′ . . . 38d′) adapted for providing N optical signals at a signal output, wherein N is greater or equal to 2.

14. Reader device (20) for an LFIA test consumable (10) according to claim 9, wherein the evaluation module (40) comprises:
a means for test result and status feedback (42); and/or
a means for wireless communication (44).

15. Process of LFIA testing comprising the steps of:
a) providing a LFIA test consumable (10) as defined in one of the claims 1 to 8, and providing a reader device (20) for a LFIA test consumable (10) as defined in one of the claims 9 to 14;
b) exposing the sample pad (15) of the LFIA test consumable (10) to a sample fluid;
c) pulling the LFIA test consumable (10) into and out of the measurement channel (32) of the reader device (20), wherein the measurement module (30) is activated and the LFIA test strips (14) of the LFIA test consumable (10) are measured;
d) evaluating at least one test result of the LFIA test consumable (10); and
e) presenting a test result and status feedback via the means for test result and status feedback (42) and/or transmitting a test result via the means for wireless communication (44).
